(19) United States
(12) Patent Application Publication
(10) Pub. No.: US 2010/0318171 A1
(43) Pub. Date: Dec. 16, 2010

(54) Multiple Stent Delivery System

(75) Inventors: Stephen Porter, Oakland, CA (US); William S. Henry, Oakland, CA (US)

Correspondence Address: VISTA IP LAW GROUP LLP, 12930 Saratoga Avenue, Suite D-2, Saratoga, CA, 95070, US

(73) Assignee: BOSTON SCIENTIFIC SCIMED, INC., Maple Grove, MN (US)

(21) Appl. No.: 12/796,562

(22) Filed: Jun. 8, 2010

Related U.S. Application Data
(60) Provisional application No. 61/186,951, filed on Jun. 15, 2009.
(60) Provisional application No. 61/186,949, filed on Jun. 15, 2009.

(51) Int. Cl. A61F 2/84

(52) U.S. Cl. 623/1.11

(57) ABSTRACT

A stent delivery system includes a delivery wire, a first stent, a second stent, and a sheath. The first stent may be disposed around a portion of a distal region of the delivery wire in a radially contracted configuration and the second stent may be disposed around a portion of the distal region of the delivery wire in a radially contracted configuration. In some cases, the first stent and the second stent may be disposed in a tandem arrangement with the first stent distal of the second stent. The sheath may be slidably disposed around the delivery wire, the first stent, and the second stent. The sheath may be retractable relative to the delivery wire to deploy the first stent and the second stent. In some embodiments, the first stent and the second stent may be sequentially deployed in an overlapping arrangement at the target site in the vessel.

### RELATED APPLICATION DATA

The present application claims the benefit under 35 U.S.C. §119 to U.S. Provisional Application Ser. Nos. 61/186,951 and 61/186,949, both filed Jun. 15, 2009. The foregoing applications are hereby incorporated by reference into the present application in their entirety.

### FIELD OF THE INVENTION

The present disclosure relates generally to medical devices and intravascular medical procedures and, more particularly, to devices and methods for delivering multiple stents to a target site in a vessel.

### BACKGROUND

The use of intravascular medical devices has become an effective method for treating many types of vascular disease. In general, one or more suitable intravascular devices are inserted into the vascular system of the patient and navigated through the vasculature to a desired target site. Using this method, virtually any target site in the patient's vascular system may be accessed, including the coronary, cerebral, and peripheral vasculature.

Medical devices such as stents, stent grafts, and vena cava filters are often utilized in combination with a delivery device for placement at a desired location within the body. A medical prosthesis, such as a stent for example, may be loaded onto a stent delivery device and then introduced into the lumen of a body vessel in a configuration having a reduced diameter. Once delivered to a target location within the body, the stent may then be expanded to an enlarged configuration within the vessel to support and reinforce the vessel wall while maintaining the vessel in an open, unobstructed condition. The stent may be configured to be self-expanding, expanded by an internal radial force such as a balloon, or a combination of self-expanding and balloon expandable.

A number of different stent delivery devices, assemblies, and methods are known, each having certain advantages and disadvantages. However, there is an ongoing need to provide alternative stent delivery devices, assemblies, and methods. In particular, there is an ongoing need to provide alternative stent delivery devices for delivering multiple stents, and methods of making and using such devices and/or assemblies.

### SUMMARY

The disclosed inventions include designs, materials, manufacturing methods, and use alternatives for various medical devices.

In one illustrative embodiment, a stent delivery system may include a delivery wire, a first stent, a second stent, and a sheath. The delivery wire may include a proximal region and a distal region. The first stent may be disposed around a portion of the distal region of the delivery wire in a radially contracted configuration. The second stent may be disposed around a portion of the distal region of the delivery wire in a radially contracted configuration. The first stent and the second stent may be disposed in a tandem arrangement with the first stent distal of the second stent. The sheath may be slidably disposed around the delivery wire, the first stent, and the second stent. The sheath may further be retractable relative to the delivery wire to deploy the first stent and the second stent. In some cases, the delivery wire may include a distal tip, and the first stent may be disposed around the distal tip and extend distally of the distal tip. In some illustrative methods, the first stent and the second stent may be sequentially deployed in an overlapping arrangement at the target site in the vessel.

The above summary of some embodiments is not intended to describe each disclosed embodiment or every implementation of the inventions disclosed herein, and the Figures, when viewed in conjunction with the following Detailed Description, will more particularly describe and exemplify these embodiments.

### BRIEF DESCRIPTION OF THE DRAWINGS

The disclosed inventions may be more completely understood in consideration of the following detailed description of various embodiments of the disclosed inventions in connection with the accompanying drawings, in which:

FIG. 1A is a flattened perspective view of an illustrative embodiment of a stent;

FIG. 1B is a flattened perspective view of an illustrative embodiment of another stent having a substantially similar cell pattern as the stent of FIG. 1A;

FIG. 1C is a flattened perspective view of an illustrative embodiment of an assembly of the stents of FIGS. 1A and 1B in an overlapping or layered configuration;

FIGS. 2A-B are flattened perspective views of an illustrative embodiment of a pair of stents having a mirrored cellular configuration;

FIG. 2C is a flattened perspective view of an illustrative embodiment of an assembly of the stents of FIGS. 2A and 2B in an overlapping or layered configuration;

FIGS. 3A-B are flattened perspective views of an illustrative embodiment of a set of helical stents;

FIG. 3C is a flattened perspective view of an illustrative embodiment of an assembly of the stents of FIGS. 3A and 3B in an overlapping or layered configuration;

FIGS. 4A-B are flattened perspective views of an illustrative embodiment of a set of stents having different cellular configurations or patterns;

FIG. 4C is a flattened perspective view of an illustrative embodiment of an assembly of the stents of FIGS. 4A and 4B in an overlapping or layered configuration;

FIGS. 5A-B are flattened perspective views of an illustrative embodiment of set of stents having a similar cell pattern with a different periodicity;

FIG. 5C is a flattened perspective view of an illustrative embodiment of an assembly of the stents of FIGS. 5A and 5B in an overlapping or layered configuration;

FIGS. 6A-B are flattened perspective views of an illustrative embodiment of a set of helical stents;

FIG. 6C is a flattened perspective view of an illustrative embodiment of an assembly of the stents of FIGS. 6A and 6B in an overlapping or layered configuration;

FIGS. 7A-B are flattened perspective views of an illustrative embodiment of a set of stents having struts at a discrete range of angles from a longitudinal axis;

FIG. 7C is a flattened perspective view of an illustrative embodiment of an assembly of the stents of FIGS. 7A and 7B in an overlapping or layered configuration;

FIGS. 8A-B are flattened perspective views of an illustrative embodiment of a set of stents having struts constructed to be within two discrete ranges of angles from the longitudinal axis;

FIG. 8C is a flattened perspective view of an illustrative embodiment of an assembly of the stents of FIGS. 8A and 8B in an overlapping or layered configuration;

FIGS. 9A-B are flattened perspective views of an illustrative embodiment of a set of stents having struts constructed to be within three discrete ranges of angles from the longitudinal axis;

FIG. 9C is a flattened perspective view of an illustrative embodiment of an assembly of the stents of FIGS. 9A and 9B in an overlapping or layered configuration;

FIG. 10A is a flattened perspective view of an illustrative embodiment of a multi-layer stent;

FIG. 10B is a perspective view of the illustrative stent of FIG. 10A in a tubular configuration;

FIGS. 10C and 10D are perspective views of the illustrative stent of FIG. 10B in a partially and completely inverted state;

FIG. 11 is a partial cross-sectional view of an illustrative stent delivery system for delivering multiple stents to a target site in a vessel;

FIGS. 12-17 are partial cross-sectional views of an illustrative procedure of deploying multiple stents in a vessel using the stent delivery system of FIG. 11;

FIG. 18 is a partial cross-sectional view of another illustrative stent delivery system for delivering multiple stents to a target site in a vessel;

FIGS. 19-24 are partial cross-sectional views of an illustrative procedure of deploying multiple stents in a vessel using the stent delivery system of FIG. 18;

FIG. 25 is a partial cross-sectional view of another illustrative stent delivery system for delivering multiple stents to a target site in a vessel;

FIGS. 26-31 are partial cross-sectional views of an illustrative procedure of deploying multiple stents in a vessel using the stent delivery system of FIG. 25; and

FIG. 32 is a partial cross-sectional view of a multiple stents deployed across an aneurysm.

While the disclosed inventions are amenable to various modifications and alternative forms, specifics thereof have been shown by way of example in the drawings and will be described in detail. It should be understood, however, that the disclosed inventions are not limited to the particular embodiments described herein or illustrated in the drawings.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the disclosed inventions.

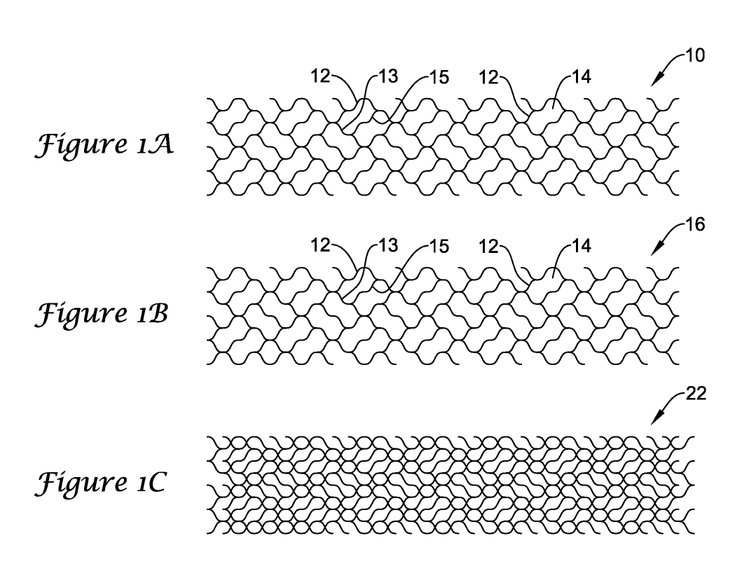

Referring now to the drawings, FIG. 1A is a flattened perspective view of an illustrative stent 10. As shown in FIG. 1A, stent 10 may have a generally cellular configuration or pattern along a length of stent 10 defined by a generally repeatable number of interconnected struts 12, connectors 13 and 15, and/or other members. The struts 12 and connectors 13 and 15 may define a number of cells 14 of stent 10. As illustrated, struts 12 may be arranged and/or configured to extend in a first general direction. Struts 12 may include a number of turns along a length of the strut and, as shown, are curved or generally s-shaped. However, it is contemplated that other patterns may be used, such as, for example zigzag patterns. A first group of connectors 13 may be arranged to extend between adjacent turns of the struts 12 and may extend in a direction generally perpendicular to struts 12. In the illustrative example, connectors 13 are shown extending between only some of the turns of struts 12, such as every other turn, to define an open cell stent. However, it is contemplated that stent 10 may be a closed cell stent, if desired. A second group of connectors 15 may be arranged to extend between adjacent turns of the connectors 13 and may extend in a direction generally parallel to struts 12. As shown, connectors 13 and 15 are curved or generally s-shaped. However, it is contemplated that other patterns may be used, such as, for example zigzag patterns.

FIG. 1B is a flattened perspective view of another illustrative stent 16 having a cellular configuration or pattern defined by a number of interconnected struts 12 and connectors 13 and 15 that is substantially similar to the cellular configuration or pattern of stent 10.

FIG. 1C is a flattened perspective view of an assembly 22 including stent 10 and stent 16 in an overlapping or layered arrangement. In the illustrative embodiment, the assembly 22 may increase the density of coverage or, in other words, decrease the porosity of the cellular configuration or pattern as compared to stent 10 or stent 16. The increase in the density of coverage may reduce the number of particles that may pass through the stent cells when in use. For example, if assembly 22 is deployed across an aneurysm in a vessel, the density of coverage of assembly 22 may effectively divert blood flow from the aneurysm to help prevent the aneurysm from rupturing.

As illustrated in FIG. 1C, stent 10 and stent 16 may be longitudinally offset so that the cellular patterns do not completely overlap. For example, stent 10 and stent 16 may be longitudinally offset by about one-half cell length. However, stent 10 and stent 16 may be offset by about one-eighth cell length, one-quarter cell length, three-quarter cell length, or any other offset length, as desired.

If, however, stent 10 and stent 16 are not offset so that there is complete strut 12 overlap due to flow in the vessel or other factors, there may be no or relatively little increase in the density of coverage. Due to the varying degrees of coverage based on the offset or alignment of stent 10 and stent 16, the assembly 22 may have a relatively low density of coverage predictability. In some situations, stents having cellular configurations or patterns differing in at least one aspect may increase the predictability of the density of coverage of the assembly. For example, and as discussed in further detail below, stents having different patterns, mirrored patterns (e.g., left-handedness, right-handedness), different periodicity of patterns, as well as stents of different constructions (e.g., tube, braid) or different materials may be used to help increase the predictability of the density of coverage or cellular porosity.

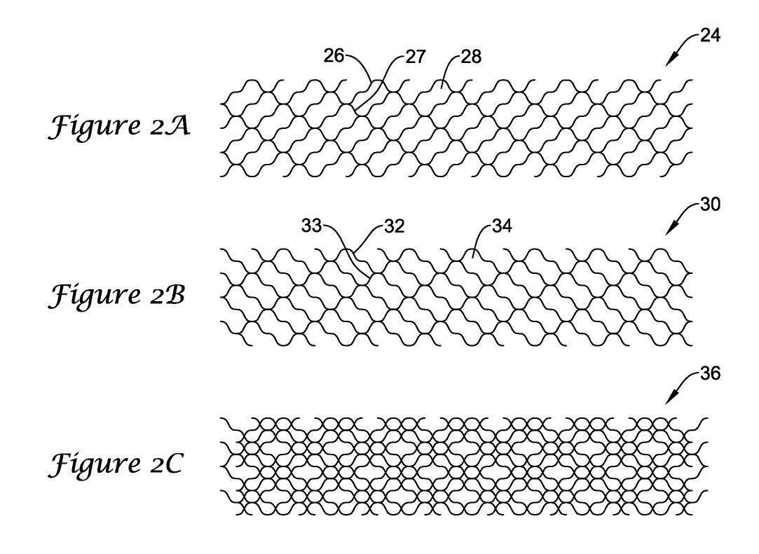

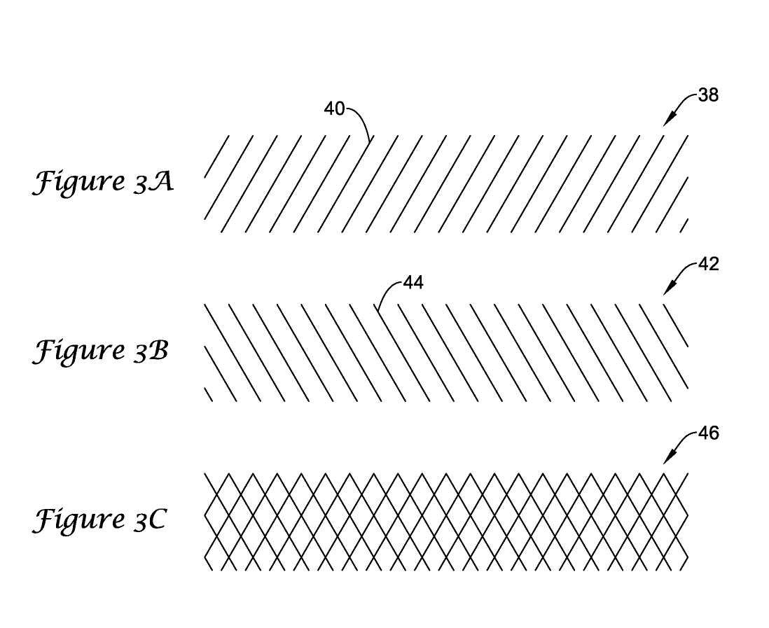

FIGS. 2A-B are flattened perspective views of an illustrative embodiment of a set of stents 24 and 30 having a mirrored cellular configuration. As illustrated in FIG. 2A, stent 24 may include a number of interconnected struts 26 and connectors 27 defining a cellular configuration or pattern. As illustrated, struts 26 and connectors 27 may define a number of cells 28. Struts 26 may be arranged and/or configured to extend in a first general direction and may include a number of turns along a length of the strut. As shown, struts 26 may be curved or generally s-shaped. However, it is contemplated that other patterns may be used, such as, for example zigzag patterns. Connectors 27 may be arranged to extend between adjacent turns of the struts 26 and may extend in a direction generally perpendicular to struts 26. In the illustrative example, connectors 27 are shown extending between only every other turn of struts 26 to define an open cell stent configuration. However, it is contemplated that stent 24 may be a closed cell stent, if desired.

As shown in FIG. 2B, stent 30 may include a number of interconnected struts 32 and connectors 33 defining a number of cells 34 having a cellular configuration similar to stent 24. However, the cellular configuration of stent 30 may be mirrored relative to stent 24. As illustrated, stent 30 may be mirrored about a longitudinal axis relative to stent 24. However, it is contemplated that stent 30 may be mirrored about a transverse axis relative to stent 24.

FIG. 2C is a flattened perspective view of an illustrative embodiment of an assembly 36 of the stents 24 and 30 of FIGS. 2A and 2B in an overlapping or layered configuration. As shown, the assembly 36 may increase the density of coverage or, in other words, decrease the porosity of the cellular configuration or pattern as compared to stent 24 or stent 30. Further, the mirrored cellular configuration or pattern may provide more predictability in the density of coverage than the assembly 22 shown in FIG. 1. For example, stent 24 and stent 30 are shown longitudinally offset. However, even if longitudinally aligned, the cellular configuration of stent 24 and stent 30 will not completely overlap.

FIGS. 3A-B are flattened perspective views of an illustrative embodiment of a set of helical stents 38 and 42. As illustrated in FIG. 3A, stent 38 may include a number of struts 40 defining a cellular configuration or pattern of stent 38. Struts 40 are shown extending in a first diagonal direction, which when rolled into a tubular stent, may be helical in shape.

As illustrated in FIG. 3B, stent 42 may include a number of struts 44 defining a cellular configuration or pattern of stent 42. Struts 44 are shown extending in a second diagonal direction, which when rolled into a tubular stent, may be helical in shape. As illustrated, struts 44 may be generally mirrored relative to struts 40. In other words, stent 38 may have a generally left-handed helical pattern or configuration and stent 42 may have a generally right-handed helical pattern or configuration.

FIG. 3C is a flattened perspective view of an illustrative embodiment of an assembly 46 of the stents 38 and 42 of FIGS. 3A and 3B in an overlapping or layered configuration. As shown, the assembly 46 may increase the density of coverage or, in other words, decrease the porosity of the cellular configuration or pattern as compared to stent 38 or stent 42. Further, the mirrored helical cellular configuration or pattern may provide more predictability in the density of coverage than the assembly 22 shown in FIG. 1. Further, relative longitudinal movement of stents 38 and 42 may not affect the density of coverage of assembly 46.

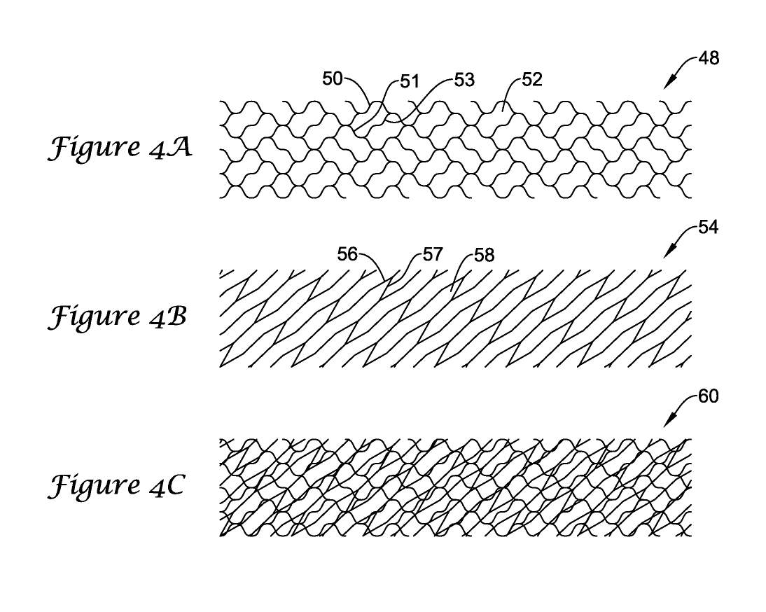

FIGS. 4A-B are flattened perspective views of an illustrative embodiment of a set of stents 48 and 54 having different cellular configurations or patterns. As illustrated in FIG. 4A, stent 48 may be defined by a generally repeatable number of interconnected struts 50 and connectors 51 and 53 defining a number of cells 52, similar to stent 10 shown in FIG. 1A.

As shown in FIG. 4B, stent 54 may include a number of interconnected struts 56 and connectors 57 defining a number of cells 58 having a cellular configuration or pattern. Struts 56 may be arranged and/or configured to extend in a first general direction and may include a number of turns along a length of the strut 56. As shown, struts 56 may be generally zigzagged. Connectors 57 may be arranged and/or configured to extend between only some of the turns of struts 56, such as every other turn, to define an open cell stent. However, it is contemplated that stent 54 may be a closed cell stent, if desired.

FIG. 4C is a flattened perspective view of an illustrative embodiment of an assembly 60 of the stents 48 and 54 of FIGS. 4A and 4B in an overlapping or layered configuration. As shown, the assembly 60 may increase the density of coverage or, in other words, decrease the porosity of the cellular configuration or pattern as compared to stent 48 or 54. Further, the different cellular configuration or patterns may provide for an assembly having a relatively higher degree of predictability as compared to assembly 22 of FIG. 1C. Further, relative longitudinal movement of stents 48 and 54 may not result in a complete overlap of stents 48 and 54.

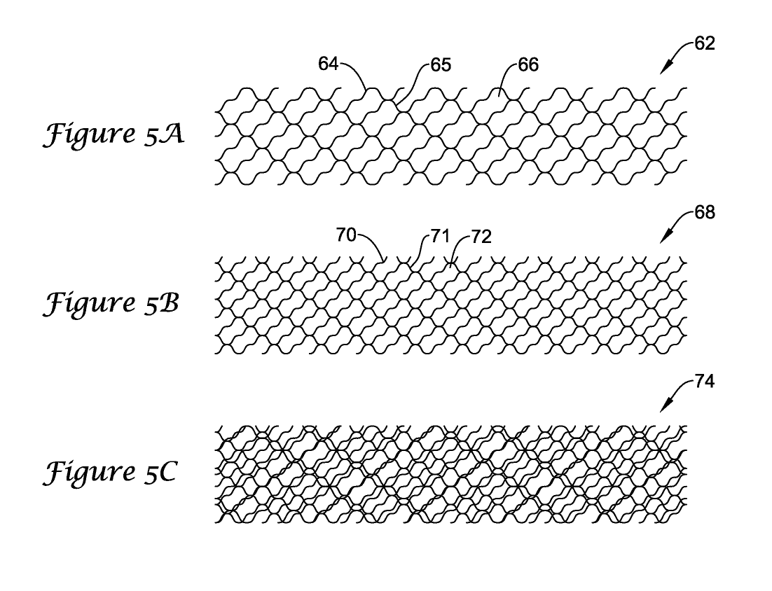

FIGS. 5A-B are flattened perspective views of an illustrative embodiment of set of stents 62 and 68 having a similar cell pattern with a different periodicity. As illustrated in FIG. 5A, stent 62 may include a number of interconnected struts 64 and connectors 65 defining a cellular configuration or pattern. As illustrated, struts 64 and connectors 65 may define a number of cells 66. Struts 64 may be arranged and/or configured to extend in a first general direction and may include a number of turns along a length of the strut. As shown, struts 64 may be curved or generally s-shaped. However, it is contemplated that other patterns may be used, such as, for example zigzag patterns. Connectors 65 may be arranged to extend between adjacent turns of the struts 64 and may extend in a direction generally perpendicular to struts 64. In the illustrative example, connectors 65 are shown extending between only every other turn of struts 64 to define an open cell stent configuration. However, it is contemplated that stent 62 may be a closed cell stent, if desired.

As shown in FIG. 5B, stent 68 may include a number of interconnected struts 70 and connectors 71 defining a number of cells 72. Stent 68 may have a cellular configuration or pattern similar to the cellular configuration of stent 62 except with a different periodicity in both the length and width directions. However, it is contemplated that the different periodicity may be only in the length or the width direction, if desired. For example, struts 70 of stent 68 may have shorter turns than struts 64 of stent 62. Further, the connectors 71 of stent 68 may be shorter than connectors 65 of stent 62.

FIG. 5C is a flattened perspective view of an illustrative embodiment of an assembly 74 of the stents 62 and 68 of FIGS. 5A and 5B in an overlapping or layered configuration. As shown, the assembly 74 may increase the density of coverage or, in other words, decrease the porosity of the cellular configuration or pattern as compared to stent 62 or 68. In assembly 74, the different periodicity may cause portions of stents 62 and stent 68 to be in phase and other portions to be out of phase. As such, any relative movement of stents 62 and 68 may not change the overall density of coverage of assembly 74. As such, assembly 74 may have a relatively higher degree of predictability as compared to assembly 22 of FIG. 1C.

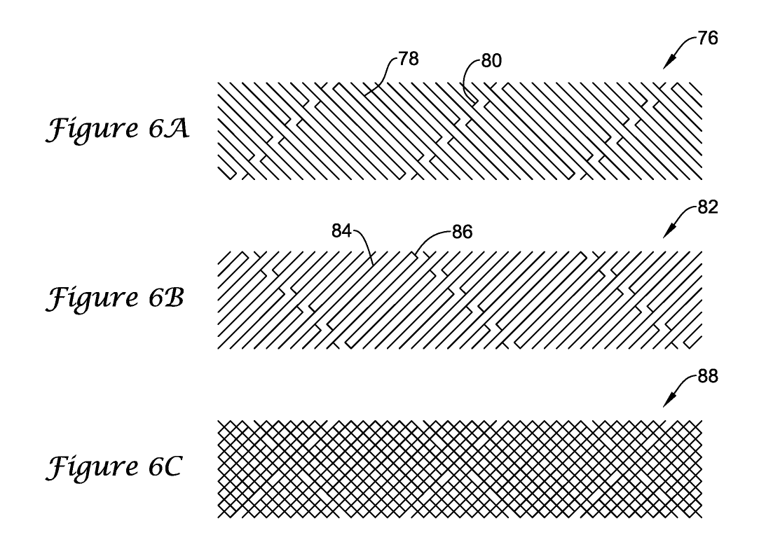

FIGS. 6A-B are flattened perspective views of an illustrative embodiment of a set of helical stents 76 and 82. As illustrated in FIG. 6A, stent 76 may include a number of interconnected struts 78 and connectors 80 defining a cellular configuration or pattern of stent 76. In some cases, the connectors 80 may help to maintain the form of the stent 76 when deployed.

As illustrated in FIG. 6B, stent 82 may include a number of interconnected struts 84 and connectors 86 defining a cellular configuration or pattern of stent 82. As illustrated, struts 84 are mirrored of struts 78. In other words, stent 76 may have a generally right-handed helical pattern or configuration and stent 82 may have a generally left-handed helical pattern or configuration.

FIG. 6C is a flattened perspective view of an illustrative embodiment of an assembly 88 of the stents 76 and 82 of FIGS. 6A and 6B in an overlapping or layered configuration. As shown, the assembly 88 may increase the density of coverage or, in other words, decrease the porosity of the cellular configuration or pattern as compared to stent 78 or 82. Further, the mirrored helical cellular configuration or pattern may provide more predictability in the density of coverage than the assembly 22 shown in FIG. 1. Further, relative longitudinal movement of stents 76 and 82 may not affect the density of coverage of assembly 88.

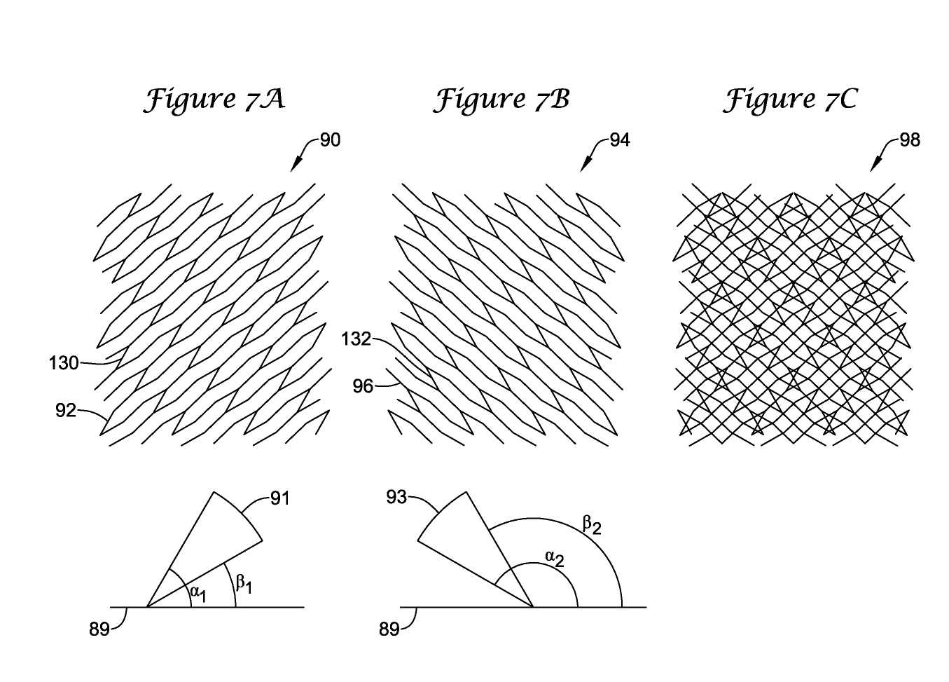

FIGS. 7-9 are flattened perspective views of illustrative embodiments of sets of stents having struts formed at discrete angles or formed by flat pattern geometry. In the illustrative stents, the stents may include struts (e.g., primary struts) at one or more discrete angles or range of angles relative to a central longitudinal axis of the stent. In some cases, the angle or range of angles of a first stent's struts may be different than the angle or range of angles of a second stent's struts so that the struts do not significantly overlap. In some cases, the stents may be configured to have one discrete angle or range of angles, two discrete angles or range of angles, three discrete angles or range of angles, four discrete angles or range of angles, five discrete angles or range of angles, or any other number of discrete angles or range of angles, as desired.

FIGS. 7A-B are flattened perspective views of an illustrative embodiment of a set of stents 90 and 94 having struts at a discrete range of angles from a longitudinal axis 89. As illustrated in FIG. 7A, stent 90 may include a number of interconnected struts 92 and connectors 130 defining a cellular configuration or pattern of the stent 90. Struts described herein may include a portion of the struts in a stent, a majority of the struts or 75% to 100% of the struts on a given stent. As shown, struts 92 and connectors 130 may be generally zigzagged in shaped, but this is not required. It is contemplated that struts 92 and connectors 130 may be generally s-shaped or any other shape, as desired. In the illustrative embodiment, stent 90 may include struts 92 configured to extend at an angle or within a range of angles from the longitudinal axis 89. The range of angles of stent 90 is shown as a vector 91 defined by angle α₁ and angle β₁. Angle α₁ may be any suitable angle, and angle β₁ may be any suitable angle different than angle α₁. The difference between angle α₁ and angle β₁ may define a size or the range of angles of vector 91. For example, the size of vector 91 may be 1 degree, 2 degrees, 3 degrees, 4 degrees, 5 degrees, 10 degrees, 15 degrees, 20 degrees, 25 degrees, 30 degrees, 35 degrees, 40 degrees, 45 degrees, or any other number of degrees, as desired. In some cases, the connectors 130 may be configured to have angles within vector 91 or angles outside of vector 91, as desired.

As illustrated in FIG. 7B, stent 94 may include a number of interconnected struts 96 and connectors 132 defining a cellular pattern of the stent 94. Struts described herein may include a portion of the struts in a stent, a majority of the struts or 75% to 100% of the struts on a given stent. As shown, struts 96 and connectors 132 may be generally in a zigzagged shape, but this is not required. It is contemplated that struts 96 and connectors 132 may be generally s-shaped or any other shape, as desired. In the illustrative embodiment, stent 94 may include struts 96 extending at an angle or range of angles from the longitudinal axis 89. The range of angles of stent 94 is shown as a vector 93 defined by angle α₂ and angle β₂. Angle α₂ may be any suitable angle not encompassed by vector 91, and angle β₂ may be any suitable angle not encompassed by vector 91 and different than angle α₂. The difference between angle α₂ and angle β₂ may define a size or the range of angles of vector 93. For example, the size of vector 93 may be 1 degree, 2 degrees, 3 degrees, 4 degrees, 5 degrees, 10 degrees, 15 degrees, 20 degrees, 25 degrees, 30 degrees, 35 degrees, 40 degrees, 45 degrees, or any other number of degrees, as desired. In some cases, the connectors 132 may be configured to have angles within vector 93 or angles outside of vector 93, as desired.

FIG. 7C is a flattened perspective view of an illustrative embodiment of an assembly 98 of the stents 90 and 94 of FIGS. 7A and 7B in an overlapping or layered configuration. As shown, the assembly 98 may increase the density of coverage or, in other words, decrease the porosity of the cellular configuration or pattern as compared to stent 90 or 94. Further, because the stents 90 and 94 having struts 92 and 96 are constructed to be within a discrete range of angles from the longitudinal axis 89, the assembly 98 may have a relatively higher degree of predictability and relatively little overlap. Further, relative longitudinal movement of stents 90 and 94 may not affect the density of coverage of assembly 98.

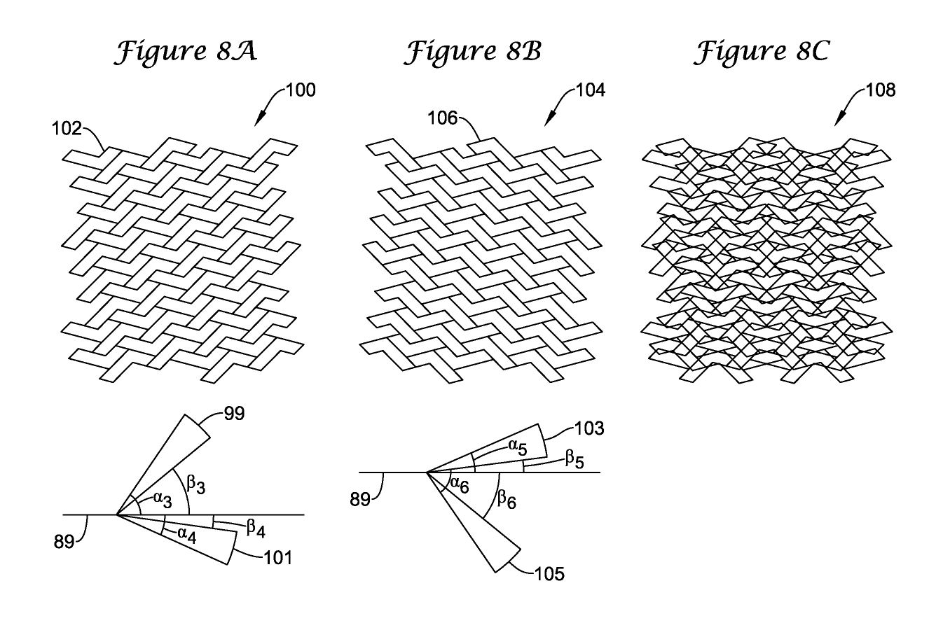

FIGS. 8A-B are flattened perspective views of an illustrative embodiment of a set of stents 100 and 104 having struts constructed to be within two discrete ranges of angles from the longitudinal axis 89. As illustrated in FIG. 8A, stent 100 may include a number of interconnected struts 102 defining a cellular pattern of the stent 100. Struts described herein may include a portion of the struts in a stent, a majority of the struts or 75% to 100% of the struts on a given stent. As shown, struts 102 may be generally zigzagged in shape, but this is not required. It is contemplated that struts 102 may be generally s-shaped or any other shape, as desired. In the illustrative embodiment, stent 100 may include struts 102 extending at two discrete angles or ranges of angles from the longitudinal axis 89. A first range of angles is shown by vector 99 defined by angle α₃ and angle β₃. A second range of angles is shown by vector 101 defined by angle α₄ and angle β₄. Angle α₃ may be any suitable angle and angle β₃ may be any suitable angle different than angle α₃. The difference between angle α₃ and angle β₃ may define a size or the range of angles of vector 99. Angle α₄ may be any suitable angle not encompassed by vector 99, and angle β₄ may be any suitable angle not encompassed by vector 99 and different than angle α₄. The difference between angle α₄ and angle β₄ may define a size or the range of angles of vector 101. Similar to vectors discussed above, vectors 99 and 101 may be any suitable size, as desired.

As illustrated in FIG. 8B, stent 104 may include a number of interconnected struts 106 defining a cellular pattern of the stent 104. As shown, struts 106 may be generally zigzagged in shape, but this is not required. Struts described herein may include a portion of the struts in a stent, a majority of the struts or 75% to 100% of the struts on a given stent. It is contemplated that struts 106 may be generally s-shaped or any other shape, as desired. In the illustrative embodiment, stent 104 may include struts 106 extending at two discrete angles or ranges of angles from the longitudinal axis 89. A first range of angles is shown by vector 103 defined by angle α₅ and angle β₅. A second range of angles is shown by vector 105 defined by angle α₆ and angle β₆. Angle α₅ may be any suitable angle not encompassed by vectors 99 and 101, and angle β₅ may be any suitable angle not encompassed by vectors 99 and 101 and different than angle α₅. The difference between angle α₅ and angle β₅ may define a size or the range of angles of vector 103. Angle α₆ may be any suitable angle not encompassed by vectors 99, 101, and 103, and angle β₆ may be any suitable angle not encompassed by vectors 99, 101, and 103 and different than angle α₄. The difference between angle α₆ and angle β₆ may define a size or the range of angles of vector 105. Similar to vectors discussed above, vectors 103 and 105 may be any suitable size, as desired.

FIG. 8C is a flattened perspective view of an illustrative embodiment of an assembly 108 of the stents 100 and 104 of FIGS. 8A and 8B in an overlapping or layered configuration. As shown, the assembly 108 may increase the density of coverage or, in other words, decrease the porosity of the cellular configuration or pattern as compared to stents 100 or 104. Further, because the stents 100 and 104 having struts 102 and 106 are constructed to be within two discrete ranges of angles from the longitudinal axis 89, the assembly 108 may have a relatively higher degree of predictability and relatively little overlap. Further, relative longitudinal movement of stents 100 and 104 may not affect the density of coverage of assembly 108.

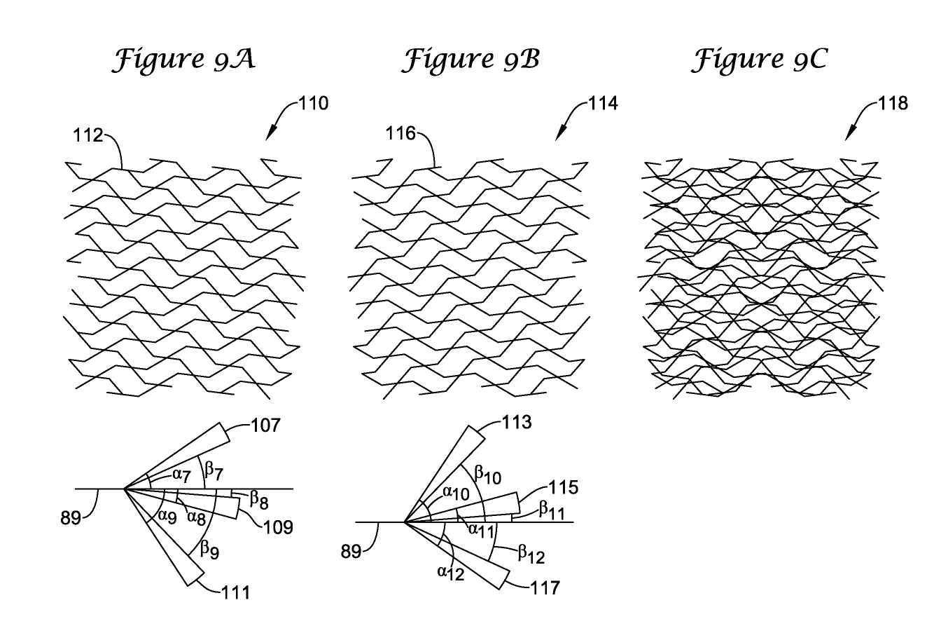

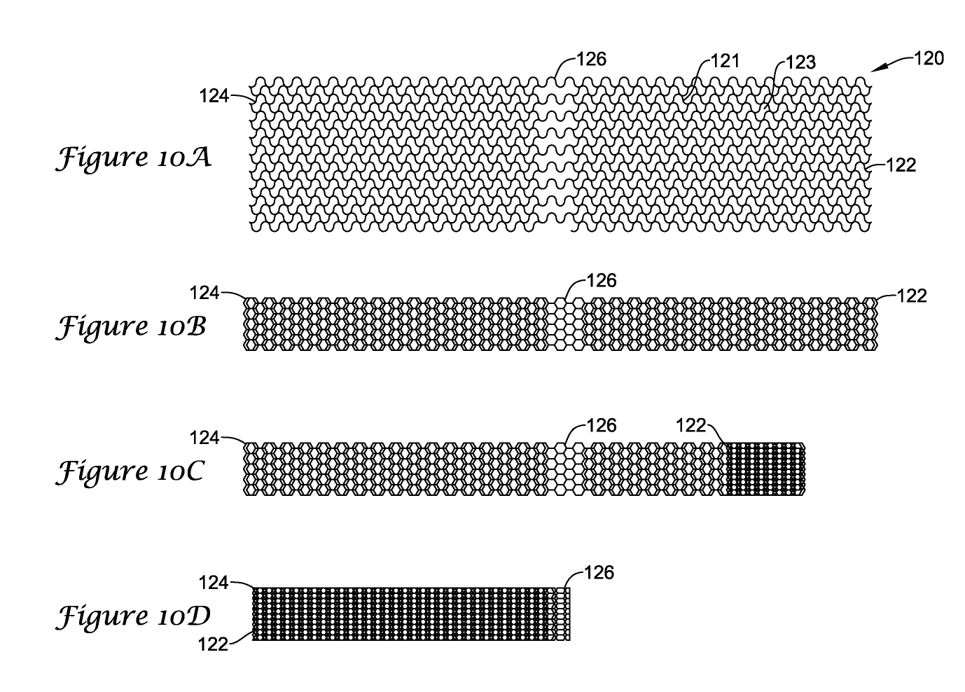

FIGS. 9A-B are flattened perspective views of an illustrative embodiment of a set of stents 110 and 114 having struts constructed to be within three discrete ranges of angles from the longitudinal axis 89. As illustrated in FIG. 9A, stent 110 may include a number of interconnected struts 112 defining a cellular configuration or pattern of the stent 110. Struts described herein may include a portion of the struts in a stent, a majority of the struts or 75% to 100% of the struts on a given stent. As shown, struts 112 may be generally zigzagged in shape, but this is not required. It is contemplated that struts 112 may be generally s-shaped or any other shape, as desired. In the illustrative embodiment, stent 110 may include stents extending at three discrete ranges of angles from the longitudinal axis 89. A first range of angles is shown by vector 107 defined by angle α₇ and angle β₇. A second range of angles is shown by vector 109 defined by angle α₈ and angle β₈. A third range of angles is shown by vector 111 defined by angle α₉ and angle β₉. Angle α₇ may be any suitable angle and angle β₇ may be any suitable angle different than angle α₇. The difference between angle α₇ and angle β₇ may define a size or the range of angles of vector 107. Angle α₈ may be any suitable angle not encompassed by vector 107 and angle β₈ may be any suitable angle not encompassed by vector 107 and different than angle α₈. The difference between angle α₈ and angle β₈ may define a size or the range of angles of vector 107. Angle α₉ may be any suitable angle not encompassed by vectors 107 and 109 and angle β₉ may be any suitable angle not encompassed by vectors 107 and 109 and different than angle α₉. The difference between angle α₉ and angle β₉ may define a size or the range of angles of vector 109. Similar to vectors discussed above, vectors 107, 109, and 111 may be any suitable size, as desired.

As illustrated in FIG. 9B, stent 114 may include a number of interconnected struts 116 defining a cellular configuration or pattern of the stent 114. As shown, struts 116 may be generally zigzagged in shape, but this is not required. Struts described herein may include a portion of the struts in a stent, a majority of the struts or 75% to 100% of the struts on a given stent. It is contemplated that struts 116 may be generally s-shaped or any other shape, as desired. In the illustrative embodiment, stent 114 may include struts 116 extending at three angles or ranges of angles from the longitudinal axis 89. A first range of angles is shown by vector 113 defined by angle α₁₀ and angle β₁₀. A second range of angles is shown by vector 115 defined by angle α₁ₗ and angle β₁₁. A third range of angles is shown by vector 117 defined by angle α₁₂ and angle β₁₂. Angle α₁₀ may be any suitable angle and angle β₁₀ may be any suitable angle not encompassed by vectors 107, 109, and 111 and different than angle α₁₁. The difference between angle α₁₀ and angle β₁₀ may define a size or the range of angles of vector 113. Angle α₁ₗ may be any suitable angle not encompassed by vectors 107, 109, 111, and 113, and angle β₁₁ may be any suitable angle not encompassed by vectors 107, 109, 111, and 113 and different than angle α₁₁. The difference between angle α₁₁ and angle β₁₁ may define a size or the range of angles of vector 115. Angle α₁₂ may be any suitable angle not encompassed by vectors 107, 109, 111, 113, and 115, and angle β₁₂ may be any suitable angle not encompassed by vectors 107, 109, 111, 113, and 115 and different than angle α₁₂. The difference between angle α₁₂ and angle β₁₂ may define a size or the range of angles of vector 117. Similar to vectors discussed above, vectors 113, 115 and 117 may be any suitable size, as desired.

FIG. 9C is a flattened perspective view of an illustrative embodiment of an assembly 118 of the stents 110 and 114 of FIGS. 9A and 9B in an overlapping or layered configuration. As shown, the assembly 118 may increase the density of coverage or, in other words, decrease the porosity of the cellular configuration or pattern as compared to stents 110 or 114. Further, because the stents 110 and 114 having struts 112 and 116 are constructed to be within three discrete ranges of angles from the longitudinal axis 89, the assembly 118 may have a relatively higher degree of predictability and relatively little overlap. Further, relative longitudinal movement of stents 110 and 114 may not affect the density of coverage of assembly 118.

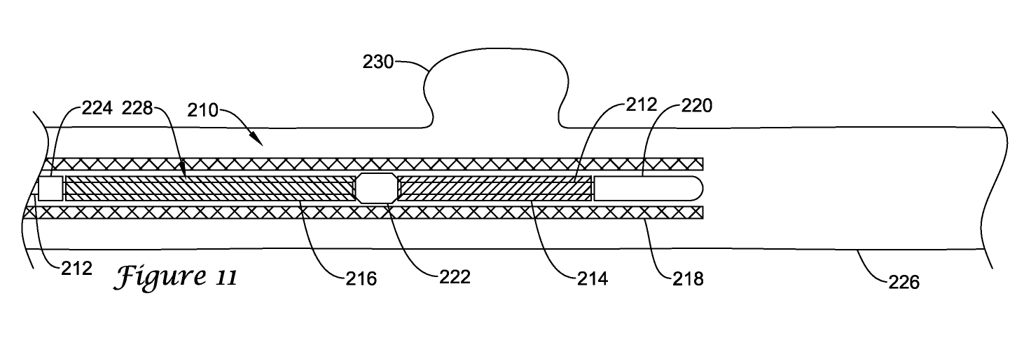

Further, it is contemplated that the foregoing stents may be deployed in an overlapping or layered arrangement or, in other cases, may be interference fit, joined, or otherwise connected to form a multi-layer stent prior to deployment, as desired. In some cases, a single layer stent may be inverted prior to assembly, during deployment, or after deployment to form a multi-layer stent. FIG. 10A is a flattened perspective view of an illustrative embodiment of a multi-layer stent 120 that can be inverted. Stent 120 may have a generally cellular configuration along the length of stent 120 defined by a generally repeatable series of interconnected struts 121. In the illustrative example, the struts 121 may define a number of cells 123 of stent 120, which form a cell pattern. As shown, the struts 121 are curved or generally s-shaped or waveform. However, it is contemplated that other patterns may be used, such as, for example, a zigzag pattern. In the illustrative example, stent 120 may include a generally central region 126 having portions of struts 121 removed. Stent 120 may also include a first end 122 and a second end 124.

FIG. 10B is a perspective view of the illustrative stent of FIG. 10A in a tubular configuration. As illustrated, stent 120 may be rolled into a tubular stent to define a first open end 122 and a second open end 124.

FIGS. 10C and 10D are perspective views of the illustrative stent of FIG. 10B in a partially and completely inverted state. As illustrated in FIG. 10C, the first end 122 of stent 120 may be partially inverted. In some cases, end 122 may be inverted into the tubular body of stent 120 or, alternatively, outside tubular body of stent 120. As shown in FIG. 10D, the stent 120 may be completely inverted so that end 122 is next to end 124. In such a configuration, stent 120 may be a multi-layer stent. Further, it is contemplated that stent 120 may include additional layers or deployed in a layered or overlapping configuration with additional stents, if desired. As illustrated, region 126 having portions of struts 121 removed may define an end of inverted stent 120. In some cases, region 126 may allow for a tighter inversion and smooth ends.

For merely illustrative purposes, the foregoing stents and assemblies have been shown in a flattened view or as a sheet. However, the stents and/or assemblies may be rolled into a generally tubular structure, similar to stent 120 shown in FIG. 10B, which may or may not have a generally varied cross-section. The tubular stent or tubular assembly may define a lumen representing the inner volumetric space bounded by the stent body. The stents or assemblies may be radially expandable from an unexpanded state to an expanded state to allow the stent to expand radially and support the vessel. In the illustrative embodiments, the stents and assemblies may be self-expanding. In this case, a sheath or other device may be used to radially constrain the stents or stent assemblies while being delivered to a treatment site within the body, but when the sheath or other device is retracted proximally from the stent or assembly, the stent may radially expand to a second configuration having a larger diameter. However, it is contemplated that the foregoing stents and/or assemblies may be expanded by an internal radial force such as a balloon, or a combination of self-expanding and balloon expandable, as desired.

Further, the foregoing stents may be constructed of any number of various materials commonly associated with medical devices. Some examples can include metals, metal alloys, polymers, metal-polymer composites, as well as any other suitable material. Examples may include stainless steels, cobalt-based alloys, pure titanium and titanium alloys, such as nickel-titanium alloys, gold alloys, platinum, and other shape memory alloys. However, it is contemplated that the foregoing stents may be constructed of any suitable material, as desired. In some cases, different stents may be constructed of different materials, if desired.

Additionally, the foregoing stents and/or assemblies may be delivered to a target site by two separate delivery systems to sequentially deliver the stents or, in other cases, by a single multiple stent delivery system. In some cases, the multiple stent delivery system may have the stents mounted thereon in an overlapping arrangement or in a tandem arrangement. However, it is contemplated that any suitable delivery system may be used, as desired.

FIGS. 11-21 are example delivery systems that may be used to deliver multiple stents, such as the stents discussed above with reference to FIGS. 1-9, to a target site in a vessel. FIG. 11 is a partial cross-sectional view of an illustrative stent delivery system 210 for delivering multiple stents 214 and 216 to a target site in a vessel 226. In the illustrative embodiment, the delivery system 210 may include a delivery member, which may be a wire or a tubular member, for example, 212 having a proximal region (not shown) and a distal region 228, two or more stents 214 and 216 disposed on a portion of the distal region 228 of the delivery wire 212 in a radially contracted configuration, and a retractable sheath 218 slidably disposed over the delivery wire 212 and/or stent 214 and 216.

Delivery wire 212 may be an elongate member having a proximal end and a distal end. In some embodiments, delivery wire 212 may be made of a conventional guidewire or may be formed of a hypotube. In either case, there are numerous materials that can be used for the delivery wire 212 to achieve the desired properties that are commonly associated with medical devices. Some examples can include metals, metal alloys, polymers, metal-polymer composites, and the like, or any other suitable material. For example, delivery wire 212 may include nickel-titanium alloy, stainless steel, a composite of nickel-titanium alloy and stainless steel. In some cases, delivery wire 212 can be made of the same material along its length, or in some embodiments, can include portions or sections made of different materials. In some embodiments, the material used to construct delivery wire 212 is chosen to impart varying flexibility and stiffness characteristics to different portions of delivery wire 212. For example, the proximal region and the distal region 228 of delivery wire 212 may be formed of different materials, for example materials having different moduli of elasticity, resulting in a difference in flexibility. For example, the proximal region can be formed of stainless steel, and the distal region 228 can be formed of a nickel-titanium alloy. However, any suitable material or combination of material may be used for delivery wire 212, as desired.

Delivery wire 212 may further include a distal tip 220, which may have an atraumatic distal end to aid in delivery wire 212 advancement. In some cases, distal tip 220 may include a coil placed over a portion of a distal end of the delivery wire 212 or, alternatively, may include a material melted down and placed over a portion of the distal end of delivery wire 212. In some cases, the distal tip 220 may include a radiopaque material to aid in visualization. Although not shown in the Figures, it is contemplated that a distal end of delivery wire 212 may include one or more tapered sections, as desired.

Delivery wire 212 may optionally include one or more bands 222 and 224 in a distal region of delivery wire 212. Bands 222 and 224 may be formed integrally into the delivery wire 212, or they may be separately formed from delivery wire 212 and attached thereto. In some cases, the bands 222 and 224 may be slidably disposed on delivery wire 212. The bands 222 and 224 may have a diameter greater than the diameter of the surrounding delivery wire 212. Bands 222 and 224 may be formed of any suitable material, such as metals, metal alloys, polymers, metal-polymer composites, and the like, or any other suitable material, as well as any radiopaque material, as desired. Alternatively, it is contemplated that the delivery wire 212 may include one or more recesses instead of providing bands 222 and 224, if desired.

In the illustrative embodiment, stents 214 and 216 may be disposed on a portion of the distal region 228 of delivery wire 212 in a radially constrained first configuration. In some cases, stents 214 and stent 216 may be disposed in a tandem arrangement. In the illustrative example, the stents 214 and 216 may be self-expanding stents. In this example, stents 214 and 216 may be radially constrained by sheath 218 while being delivered to a treatment site within the body, but when sheath 218 is retracted proximally, stents 214 and 216 may radially expand to a second configuration having a larger diameter.

Each of stents 214 and 216 may be constructed of a plurality of interconnected struts, connectors, or other members to define a stent pattern. In the illustrative example, the stents 214 and 216 may include struts configured in a helical pattern where stents 214 and 216 have opposite orientations. However, it is contemplated that any stent disclosed herein or any combination of stent disclosed herein may be used, as well as any other suitable stents, as desired.

As illustrated in FIG. 11, stent 216 may be disposed distal of band 224 and proximal of band 222. Stent 214 may be disposed distal of band 222 but proximal of the distal tip 220. In some embodiments, the distal tip 220 may have a diameter greater than the delivery wire 212, but this is not required. The bands 222 and 224 and distal tip 220 may be configured to engage the proximal and distal ends of stents 214 and 216 to prevent slippage between the delivery wire 212 and stents 214 and 216 when moving the delivery wire 212 relative to sheath 218.

Sheath 218 may be an elongate tubular member that may have a distal region or end that is slidably disposed over delivery wire 212, having an annular space sufficient in size to receive radially contracted stents 214 and 216 therein. In the illustrative embodiment, movement of sheath 218 in a proximal direction relative to delivery wire 212 may expose stents 214 and/or 216, allowing expansion of stents 214 and/or 216. There are numerous materials that can be used for the sheath 218 to achieve the desired properties that are commonly associated with medical devices. Some examples can include metals, metal alloys, polymers, metal-polymer composites, and the like, or any other suitable material. Examples of suitable metals and metal alloys can include stainless steel, such as 304V, 304L, and 316L stainless steel; nickel-titanium alloy such as a superelastic (i.e., pseudoelastic) or linear elastic nitinol; nickel-chromium alloy; nickel-chromium-iron alloy; cobalt alloy; tungsten or tungsten alloys; tantalum or tantalum alloys, gold or gold alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); or the like; or other suitable metals, or combinations or alloys thereof. Examples of some suitable polymers can include, but are not limited to, polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro(propyl vinyl ether) (PFA), polyether-ester, polymer/metal composites, or mixtures, blends or combinations thereof. Sheath 218 can optionally be lined on an inner surface, an outer surface, or both with a lubricious material, if desired.

As shown in FIG. 11, the stent delivery system 210 may be positioned in the vessel 226 so that stent 214 is positioned adjacent to the target site, which in the illustrative example is a weakened region of the vessel 226 or an aneurysm 230. In some cases, stents 214 and 216 may be configured to be deployed across the aneurysm 230 to help divert blood flow in the vessel 226 from entering the aneurysm 230. However, this treatment site is merely illustrative and is not meant to be limiting in any manner. It is contemplated that the delivery system 210 may be used to deliver multiple stents to a target site, such as a stenoses or other target site, as desired.

In some cases, the sheath 218 and delivery wire 212 with radially contracted stents 214 and 216 may be advanced to the target site, or aneurysm 230, as an assembly. In these cases, the stent delivery system 210 may optionally be inserted into a proximal end of an introducer or other catheter and subsequently advanced to the aneurysm 230. In other cases, the sheath 218 may be advanced to the target site first and then the delivery wire 212 with radially contracted stents 214 and 216 may be inserted into a proximal end of sheath 218 and advanced through the sheath lumen to the target site.

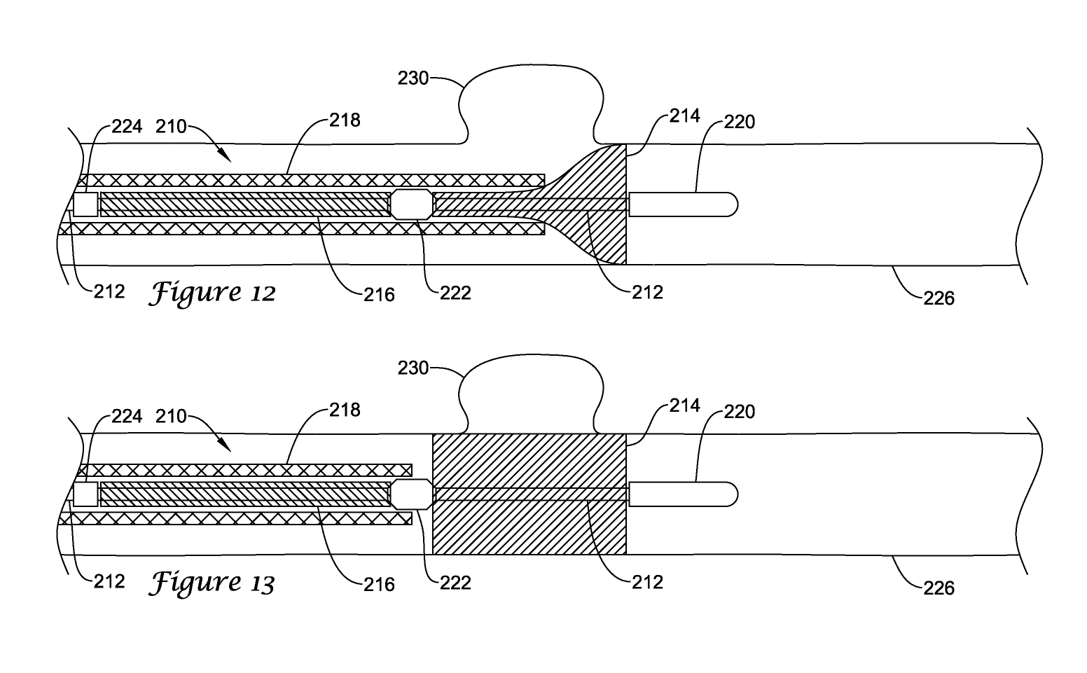

FIGS. 12-17 are partial cross-sectional views of an illustrative procedure for sequentially deploying the two or more stents 214 and 216 in vessel 226 in an overlapping arrangement using the stent delivery system 210 of FIG. 11. After the stent delivery system 210 has been positioned so that stent 214 is aligned with aneurysm 230, as shown in FIG. 12, sheath 218 may be partially retracted from the delivery wire 212 exposing a distal portion of stent 214. As illustrated, when self-expanding stent 214 is exposed, stent 214 radially expands to engage a portion of the vessel 226 wall.

As illustrated in FIG. 13, continued retraction of sheath 218 relative to delivery wire 212 to a position proximal of stent 214 completely deploys stent 214. As stent 214 is deployed, stent 214 fully expands and engages the vessel 226 wall on both sides of aneurysm 230.

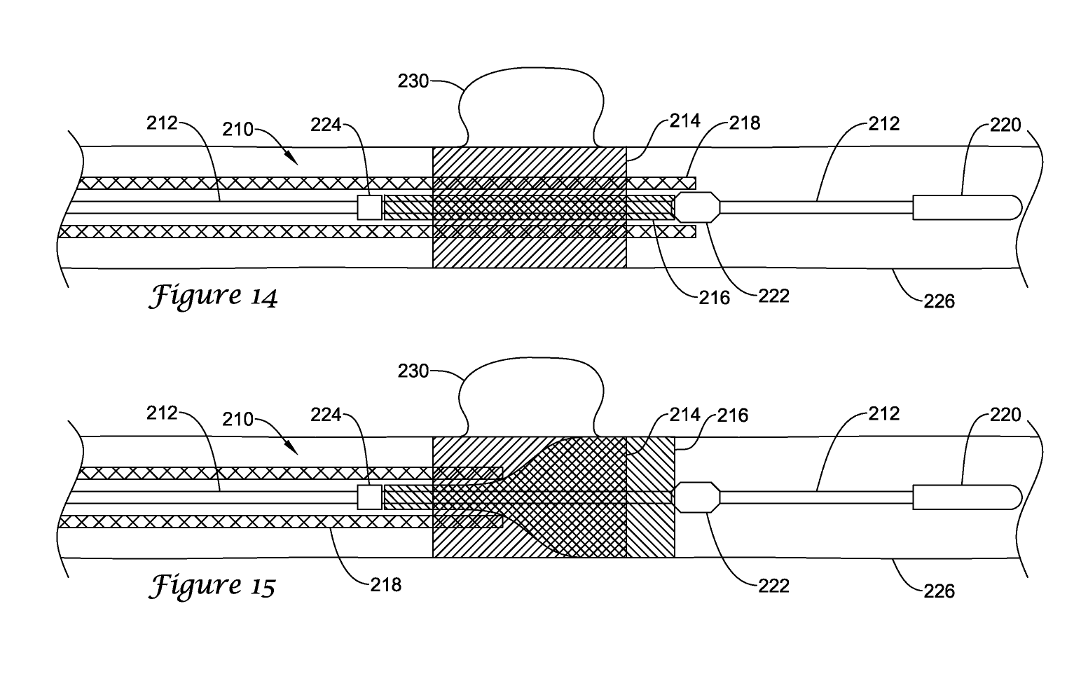

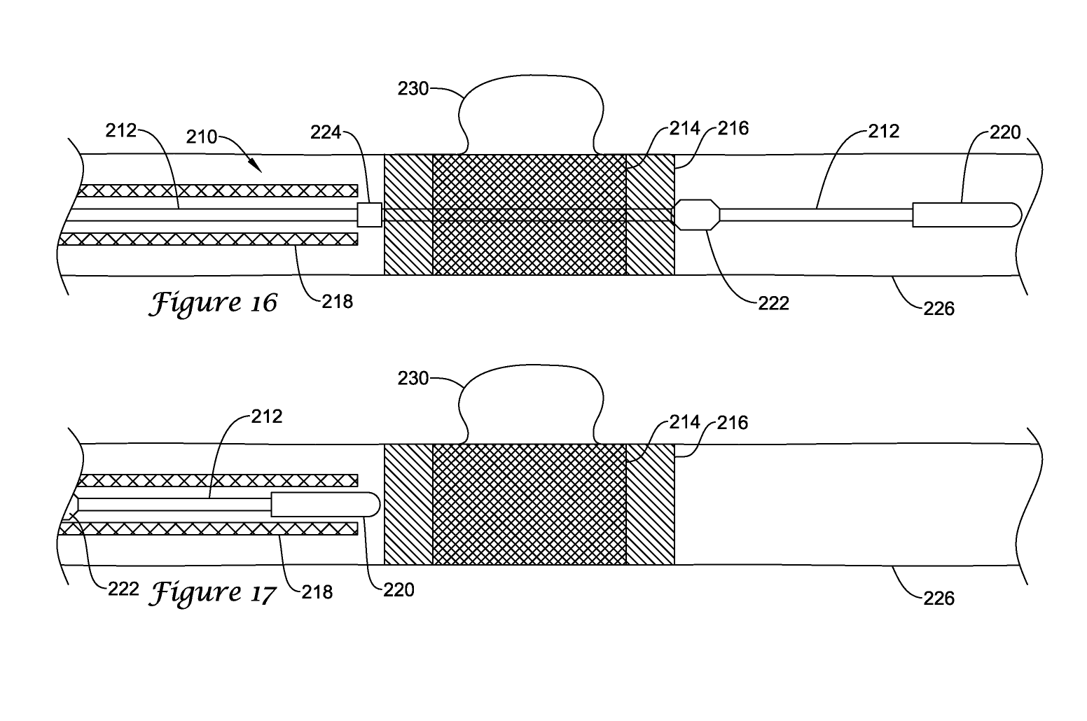

With stent 214 deployed, as illustrated in FIG. 14, sheath 218 and delivery wire 212 including radially contracted stent 216 may be advanced distally through the lumen of stent 214 until stent 216 is in a desired alignment with stent 214. In some cases, the alignment may be partially overlapping or completely overlapping, as desired. Then, as shown in FIG. 15, sheath 218 may once again be retracted relative to delivery wire 212 exposing the distal portion of stent 216. When the distal portion of stent 216 is no longer constrained by sheath 218, distal portion of stent 216 may radially expand. As shown in FIG. 16, sheath 218 may be further retracted to a position proximal of stent 216. In this position, stent 216 is no longer radially constrained and may radially expand and engage stent 214 and vessel wall 226.

After both stents 214 and 216 are deployed across the aneurysm 230, as shown in FIG. 17, delivery wire 212 may be optionally retracted into sheath 218. Then, delivery wire 212 and sheath 218 may be withdrawn from the vessel 226 together or separate, as desired. In the illustrative embodiment, stent 216 is shown as having a length greater than stent 214. However, it is contemplated that stent 214 and stent 216 may be the same length or that stent 214 may be longer than stent 216, as desired. Further, it is contemplated that stents 214 and 216 may be deployed in a completely overlapping configuration, in a non-overlapping configuration, or any partially overlapping configuration, as desired.

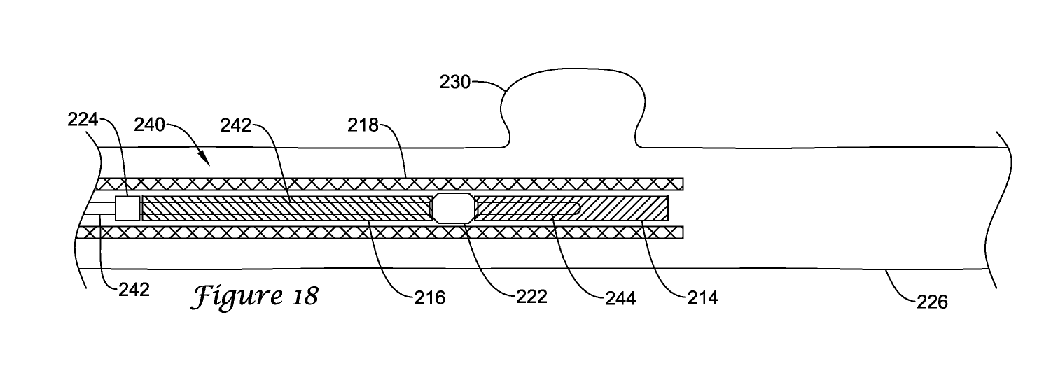

FIG. 18 is partial cross-sectional view of another illustrative stent delivery system 240 for delivering multiple stents 214 and 216 to a target site in a vessel 226. In the illustrative embodiment, stent delivery system 240 may include a delivery member 242, which may be a wire or tubular member, for example, two or more stents 214 and 216 disposed on delivery wire 242, and a sheath 218 slidably disposed around delivery wire 242. In the illustrative example, stent 214, stent 216, and sheath 218 may be similar to those discussed above with reference to stent delivery system 210. Further, delivery wire 242 may be similar to delivery wire 212 in many respects. However, delivery wire 242 may be configured to have a shortened distal region and have fewer bands 222 or other diametric changes.

As illustrated in FIG. 18, delivery wire 242 may include a distal tip 244 similar to distal tip 220. However, distal tip 244 may be disposed adjacent to the distal end of band 222. In this embodiment, stent 214 may be disposed about distal tip 244 and may extend distally thereof. In this embodiment, band 222 may be provided proximally of stent 214 to engage the proximal end of stent 214 for pushability, but since stent 214 extends distally of the delivery wire 242, there may be no diametric change distal of stent 214 to provide pullability.

As shown in FIG. 18, the stent delivery system 240 may be positioned in the vessel 226 so that stent 214 is positioned adjacent to the target site, which in the illustrative example is a weakened region of the vessel 226 or an aneurysm 230. In some cases, stents 214 and 216 may be configured to be deployed across the aneurysm 230 to help divert blood flow in the vessel 226 from entering the aneurysm 230. However, this treatment site is merely illustrative and is not meant to be limiting in any manner. It is contemplated that the delivery system 210 may be used to deliver multiple stents to a target site or multiple sites, such as a stenoses or other target site, as desired.

In some cases, the sheath 218 and delivery wire 242 with radially contracted stents 214 and 216 may be advanced to the target site, or aneurysm 230, as an assembly. In these cases, the stent delivery system 240 may optionally be inserted into a proximal end of an introducer or other catheter and subsequently advanced to the aneurysm 230. In other cases, the sheath 218 may be advanced to the target site first and then the delivery wire 242 with radially contracted stents 214 and 216 may be inserted into a proximal end of sheath 218 and advanced through the sheath lumen to the target site.

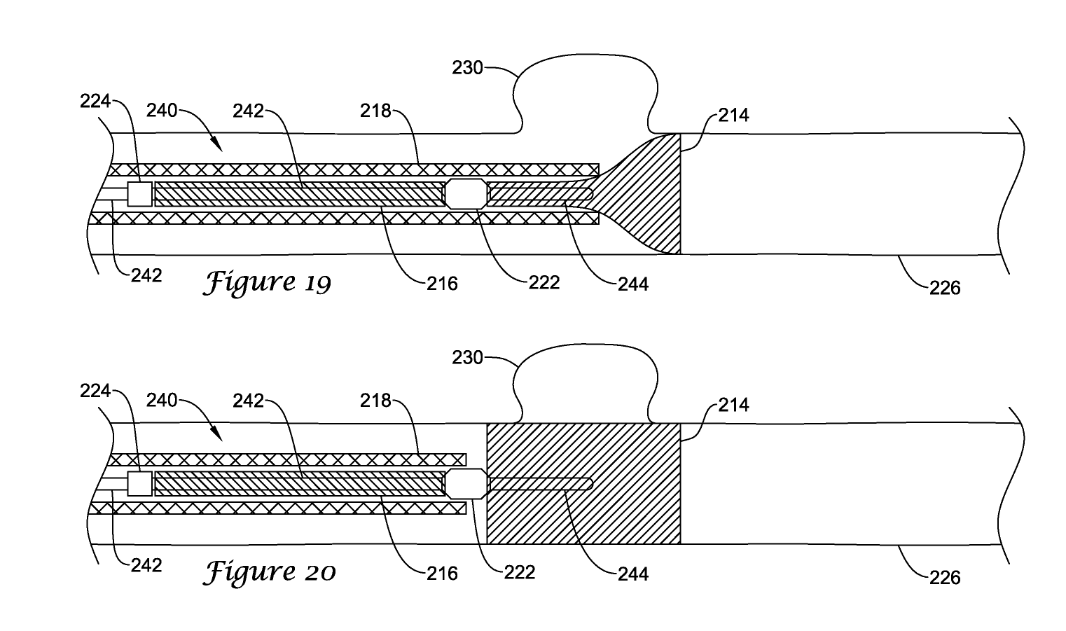

FIGS. 19-24 are partial cross-sectional views of an illustrative procedure for sequentially deploying the two or more stents 214 and 216 in vessel 226 in an overlapping arrangement using the stent delivery system 240 of FIG. 18. After the stent delivery system 240 has been positioned so that stent 214 is aligned with aneurysm 230, as shown in FIG. 19, sheath 218 may be partially retracted from the delivery wire 242, exposing a distal portion of stent 214. As illustrated, when self-expanding stent 214 is exposed, stent 214 radially expands to engage a portion of the vessel 226 wall.

As illustrated in FIG. 20, continued retraction of sheath 218 relative to delivery wire 242 to a position proximal of stent 214 completely deploys stent 214. As stent 214 is deployed, stent 214 fully expands and engages the vessel 226 wall on both sides of aneurysm 230.

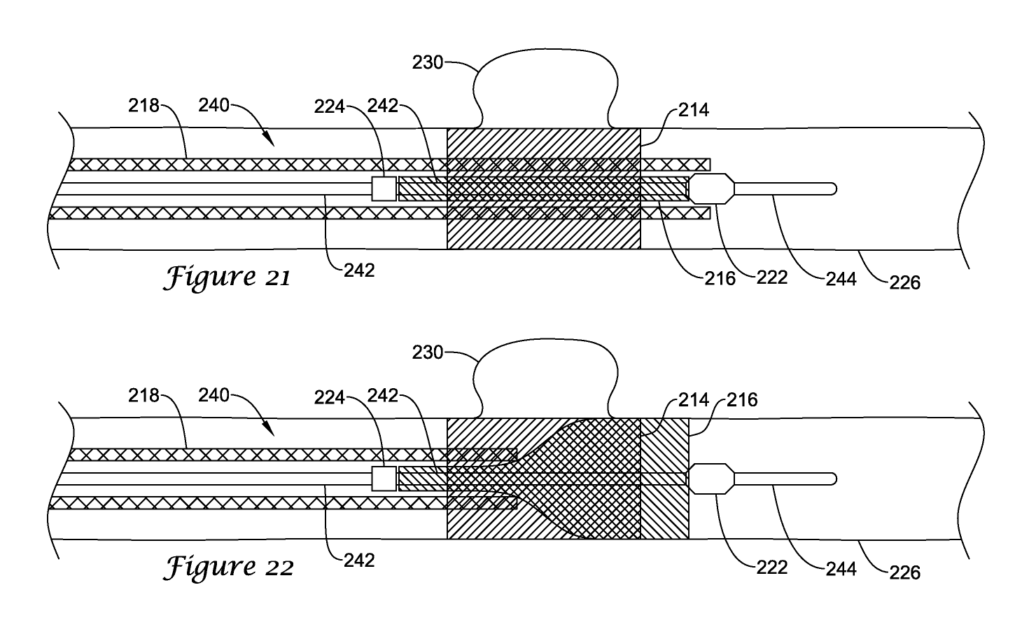

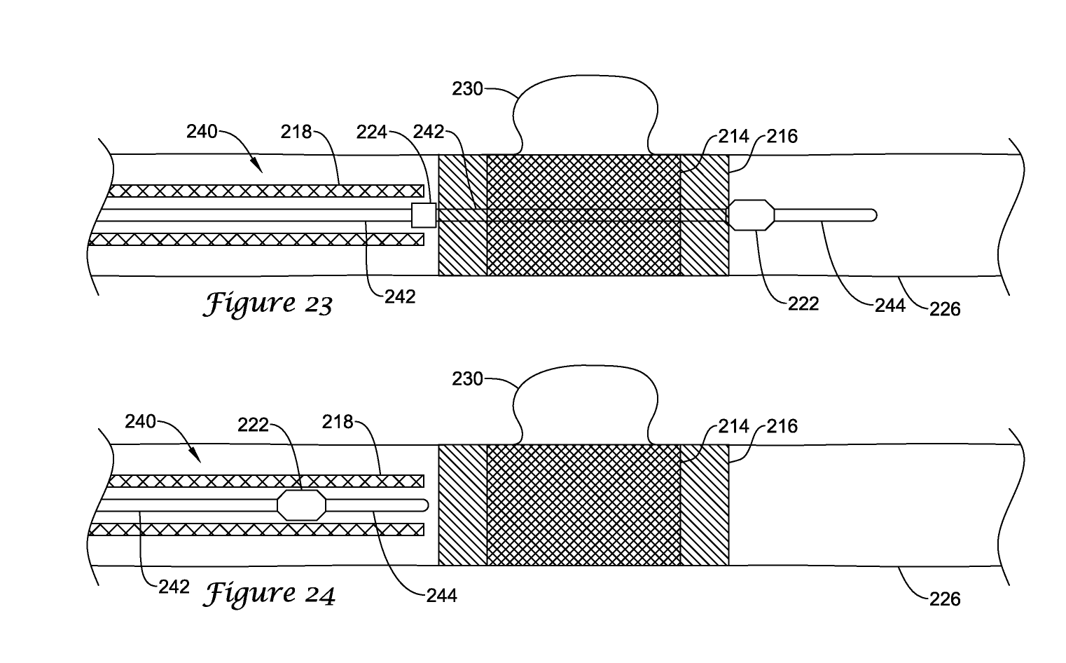

With stent 214 deployed, as illustrated in FIG. 21, sheath 218 and delivery wire 242 including radially contracted stent 216 may be advanced distally through the lumen of stent 214 until stent 216 is in a desired alignment with stent 214. In some cases, the alignment may be partially overlapping or completely overlapping, as desired. Then, as shown in FIG. 22, sheath 218 may once again be retracted relative to delivery wire 242, exposing the distal portion of stent 216. When the distal portion of stent 216 is no longer constrained by sheath 218, distal portion of stent 216 may radially expand. As shown in FIG. 23, sheath 218 may be further retracted to a position proximal of stent 216. In this position, stent 216 is no longer radially constrained and may radially expand and engage stent 214 and vessel wall 226.

After both stents 214 and 216 are deployed across the aneurysm 230, as shown in FIG. 24, delivery wire 242 may be optionally retracted into sheath 218. Then, delivery wire 242 and sheath 218 may be withdrawn from the vessel 226 together or separate, as desired. In the illustrative embodiment, stent 216 is shown as having a length greater than stent 214. However, it is contemplated that stent 214 and stent 216 may be the same length or that stent 214 may be longer than stent 216, as desired. Further, it is contemplated that stents 214 and 216 may be deployed in a completely overlapping configuration, in a non-overlapping configuration, or any partially overlapping configuration, as desired.

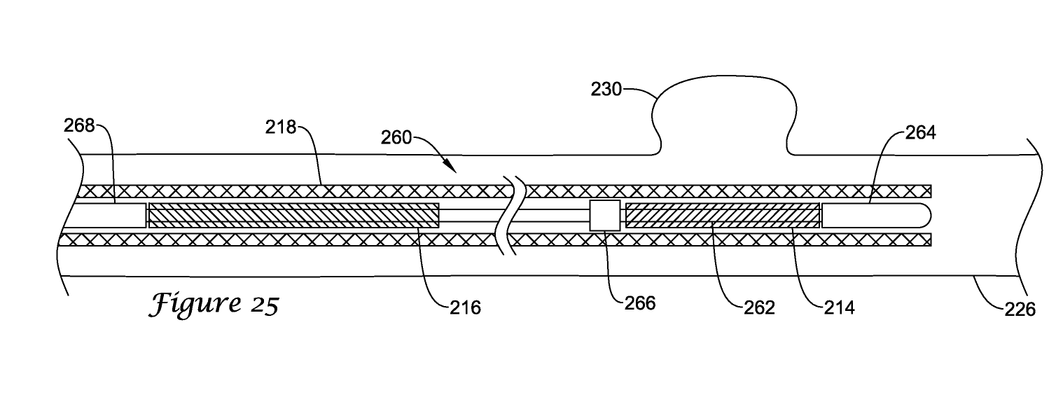

FIG. 25 is partial cross-sectional view of another illustrative stent delivery system 260 for delivering multiple stents 214 and 216 in a vessel 226. In the illustrative embodiment, the stent delivery system 260 may include a delivery wire 262, two or more radially contracted stents 214 and 216 disposed about the delivery wire 262, a sheath 218 slidably disposed over delivery wire 262, and a push sheath 268 slidably disposed between delivery wire 262 and sheath 218. In the illustrative example, stent 214, stent 216, and sheath 218 may be similar to those discussed above with reference to stent delivery system 210.

In the illustrative embodiment, delivery wire 262 may be similar to delivery wire 212, shown in FIG. 11. However, in this illustrative embodiment, delivery wire 262 may include only one band 266 that may be slidable over delivery wire 262. Further, stent 216 may be disposed at a location spaced proximal of stent 214.

Push sheath 268 may be a tubular member having a proximal end, a distal end, and a lumen extending therebetween. In some cases, push sheath 268 may be slidably disposed about delivery wire 262 and within sheath 218. Push sheath 268 may be configured to slide along delivery wire 262 and engage a proximal end of stent 216 and slide stent 216 distally relative to delivery wire 262, as will be discussed in further detail below. There are numerous materials that can be used for push sheath 268 to achieve the desired properties that are commonly associated with medical devices. Some examples can include metals, metal alloys, polymers, metal-polymer composites, and the like, or any other suitable material. Examples of suitable metals and metal alloys can include stainless steel, such as 304V, 304L, and 316L stainless steel; nickel-titanium alloy such as a superelastic (i.e., pseudoelastic) or linear elastic nitinol; nickel-chromium alloy; nickel-chromium-iron alloy; cobalt alloy; tungsten or tungsten alloys; tantalum or tantalum alloys, gold or gold alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); or the like; or other suitable metals, or combinations or alloys thereof. Examples of some suitable polymers can include, but are not limited to, polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro(propyl vinyl ether) (PFA), polyether-ester, polymer/metal composites, or mixtures, blends or combinations thereof. Push sheath 268 can optionally be lined on an inner surface, an outer surface, or both with a lubricious material, if desired.

As shown in FIG. 25, the stent delivery system 260 may be positioned in the vessel 226 so that stent 214 is positioned adjacent to the target site, which in the illustrative example is a weakened region of the vessel 226 or an aneurysm 230. In some cases, stents 214 and 216 may be configured to be deployed across the aneurysm 230 to help divert blood flow in the vessel 226 from entering the aneurysm 230. However, this treatment site is merely illustrative and is not meant to be limiting in any manner. It is contemplated that the delivery system 210 may be used to deliver multiple stents to a target site or multiple sites, such as a stenoses or other target site, as desired.

In some cases, the sheath 218, push sheath 268, and/or delivery wire 262 with radially contracted stents 214 and 216 may be advanced to the target site, or aneurysm 230, as an assembly. In these cases, the stent delivery system 260 may optionally be inserted into a proximal end of an introducer or other catheter and subsequently advanced to the aneurysm 230. In other cases, the sheath 218 may be advanced to the target site first, and then push sheath 268 and the delivery wire 262 with radially contracted stents 214 and 216 may be inserted into a proximal end of sheath 218 and advanced through sheath lumen to the target site.

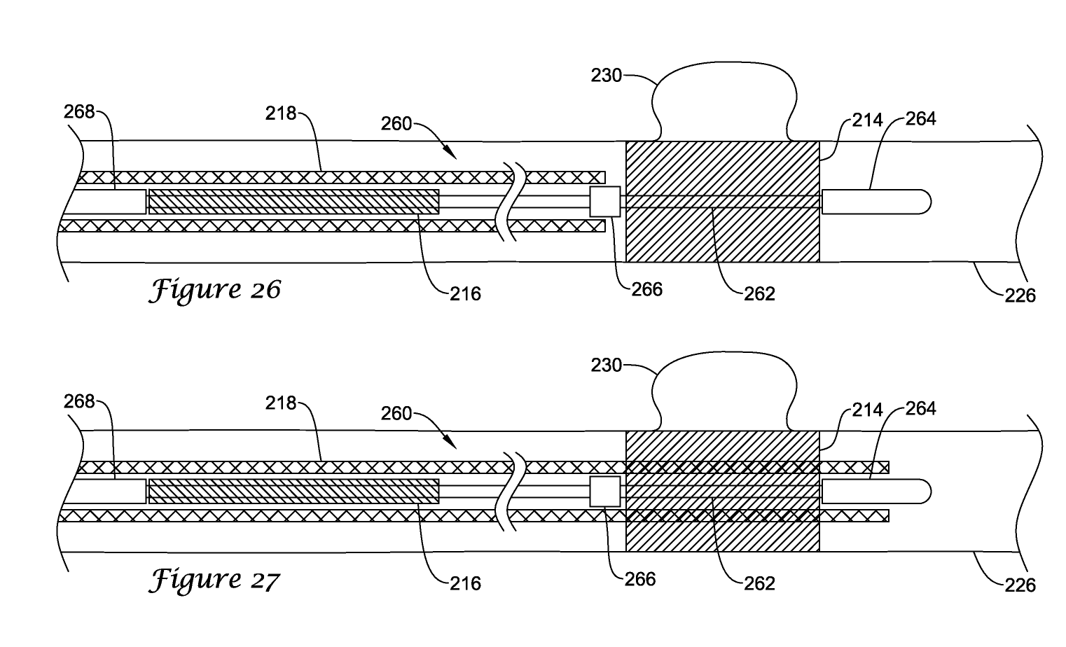

FIGS. 26-31 are partial cross-sectional views of an illustrative procedure for sequentially deploying the two or more stents 214 and 216 in vessel 226 in an overlapping arrangement using the stent delivery system 260 of FIG. 25. After the stent delivery system 260 has been positioned so that stent 214 is aligned with aneurysm 230, as shown in FIG. 26, sheath 218 may be retracted from the delivery wire 262 to a position proximal of stent 214, completely deploying stent 214. As stent 214 is deployed, stent 214 fully expands and engages the vessel 226 wall on both sides of aneurysm 230.

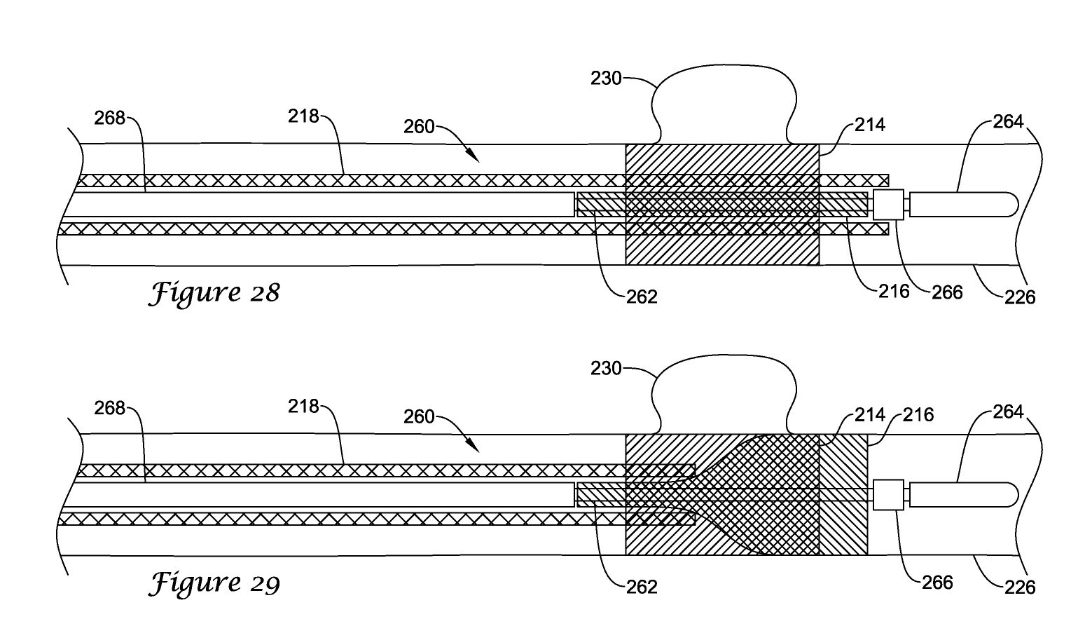

With stent 214 deployed, as illustrated in FIG. 27, sheath 218 may be advanced distally through the lumen of stent 214 until the distal end of sheath 218 is positioned adjacent distal tip 264 of delivery wire 262. Then, as shown in FIG. 28, push sheath 268 may be advanced distally relative to delivery wire 262. As push sheath 268 is advanced distally, push sheath 268 slides stent 216 distally along delivery wire 262. A distal end of stent 216 may engage band 266 and slide band 266 distally along the delivery wire 262 to a position adjacent distal tip 264. The push sheath 268 may be advanced until stent 216 is in a desired alignment with stent 214.

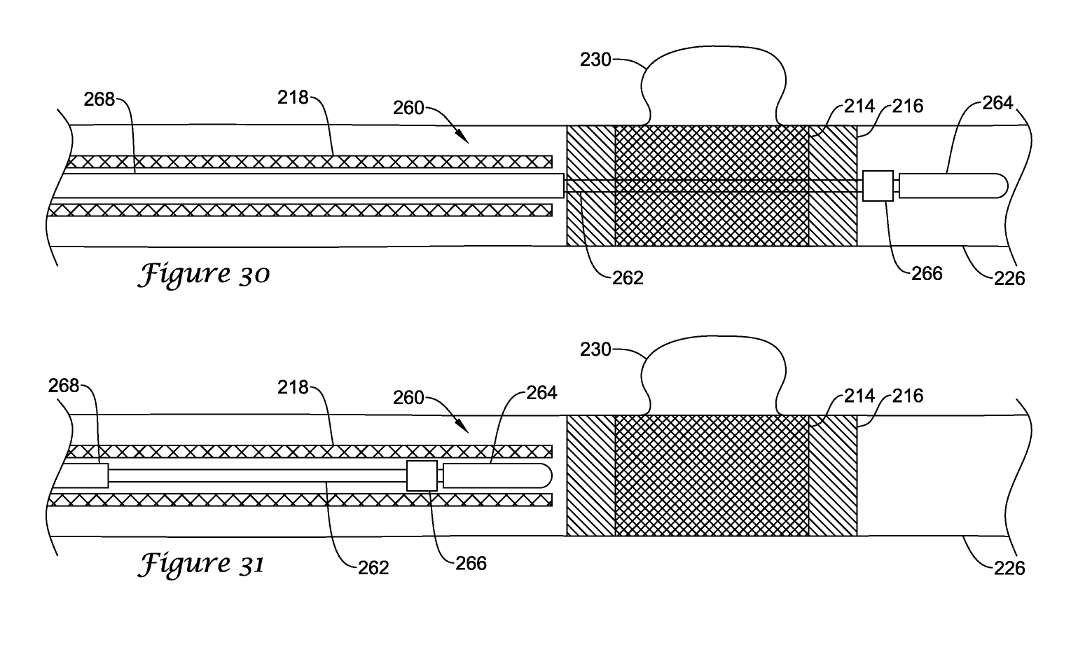

Then, as shown in FIG. 29, sheath 218 may once again be retracted relative to delivery wire 262 exposing the distal portion of stent 216. When the distal portion of stent 216 is no longer constrained by sheath 218, distal portion of stent 216 may radially expand. As shown in FIG. 30, sheath 218 may be further retracted to a position proximal of stent 216. In this position, stent 216 is no longer radially constrained and may radially expand and engage stent 214 and vessel wall 226.

After both stents 214 and 216 are deployed across the aneurysm 230, as shown in FIG. 31, delivery wire 262 may be optionally retracted into sheath 218. Then, delivery wire 262, sheath 218, and push sheath 268 may be withdrawn from the vessel 226 together or separate, as desired.

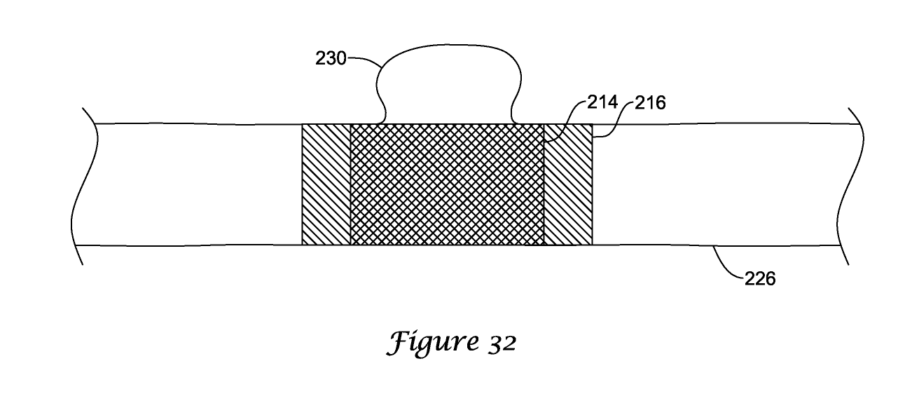

FIG. 32 is a partial cross-sectional view of a multiple stents 214 and 216 deployed across an aneurysm 230. As illustrated, stent 216 is shown as having a length greater than stent 214. However, it is contemplated that stent 214 and stent 216 may be the same length or that stent 214 may be longer than stent 216, as desired. Further, it is contemplated that stents 214 and 216 may be deployed in a completely overlapping configuration, in a non-overlapping configuration, or any partially overlapping configuration, as desired.

For merely illustrative purposes, the foregoing delivery systems 210, 240, and 260 have been described with reference to two stents. However, this is not meant to be limiting in any manner. It is contemplated that any suitable number of stents may be used with the illustrative stent delivery systems 210, 240, and 260, as desired. Furthermore, for mere simplicity, the foregoing disclosure has been described with reference to stents. However, this is not meant to be limiting in any manner. It is contemplated that grafts, stent-grafts, vena cava filters, expandable frameworks, and/or other radially expandable endoprostheses may be used, as desired.

In at least some embodiments, portions or all of the stent delivery systems 210, 240, and/or 260, or other components that are part of or used in the systems, may be doped with, made of, or otherwise include a radiopaque material. Radiopaque materials are understood to be materials capable of producing a relatively bright image on a fluoroscopy screen or another imaging technique during a medical procedure. This relatively bright image aids the user of devices in determining its location. Some examples of radiopaque materials can include, but are not limited to, gold, platinum, palladium, tantalum, tungsten alloy, polymer material loaded with a radiopaque filler, and the like. Additionally, radiopaque marker bands and/or coils may be incorporated into the design of stent delivery systems 210, 240, and/or 260 to achieve the same result.

In some embodiments, a degree of MRI compatibility is imparted into catheters. For example, to enhance compatibility with Magnetic Resonance Imaging (MRI) machines, it may be desirable to make the stent delivery systems 210, 240, and/or 260, or other portions of the stent delivery systems 210, 240, and/or 260, in a manner that would impart a degree of MRI compatibility. For example, elongated members 212, 242, and/or 262, sheath 218, stents 214 and 216, push sheath 268, or other portions of stent delivery systems 210, 240, and/or 260, may be made of a material that does not substantially distort the image and create substantial artifacts (artifacts are gaps in the image). Certain ferromagnetic materials, for example, may not be suitable because they may create artifacts in an MRI image. Stent delivery systems 210, 240, and/or 260, or portions thereof, may also be made from a material that the MRI machine can image. Some materials that exhibit these characteristics include, for example, tungsten, Elgiloy, MP35N, nitinol, and the like, and others. In some embodiments, a sheath and/or coating, for example a lubricious, a hydrophilic, a protective, or other type of material may be applied over portions or all of the stent delivery systems 210, 240, and/or 260, or other portions of the systems.

The disclosed inventions should not be considered limited to the particular examples described above. Various modifications, equivalent processes, as well as numerous structures to which the disclosed inventions may be applicable will be readily apparent to those of skill in the art to which the inventions are directed upon review of the instant specification. Furthermore, it is contemplated that the various features and components of the foregoing embodiments can be mixed and matched as desired. Changes may be made in details, particularly in matters of shape, size, and arrangement of steps without exceeding the claimed scope of the disclosed inventions, which is, of course, defined by the appended claims.

What is claimed is:

1. A method for deploying two or more stents at a target site in a vessel, the method comprising:
providing a stent delivery device including an elongate member, two or more self-expanding stents, and a sheath, the elongate member having a proximal region and a distal region, the two or more self-expanding stents being disposed in a radially contracted configuration around a portion of the distal region of the elongate member, and the sheath being slidably disposed around the elongate member and the two or more stents; and
sequentially deploying the two or more self-expanding stents in an overlapping arrangement at the target site in the vessel.

2. The method of claim 1, wherein sequentially deploying the two or more self-expanding stents in an overlapping arrangement at the target site in the vessel comprises:
retracting the sheath relative to the elongate member to a position proximal of a first self-expanding stent of the two or more self-expanding stents to deploy the first self-expanding stent;
moving the elongate member and the sheath relative to the first self-expanding stent to at least partially align a second self-expanding stent of the two or more stents with the first self-expanding stent; and
retracting the sheath relative to the elongate member to a position proximal of the second self-expanding stent to deploy the second self-expanding stent in an overlapping arrangement with the first self-expanding stent.

3. The method of claim 1, further comprising advancing the stent delivery system to position a first self-expanding stent of the two or more self-expanding stents adjacent to the target site in the vessel.

4. The method of claim 1, wherein a first self-expanding stent of the two or more self-expanding stents is disposed about the distal end of the elongate member and extends distally of the elongate member.

5. The method of claim 1, wherein the elongate member includes one or more radially expanded portions.

6. The method of claim 5, wherein a first radially expanded portion is provided proximal of both a first stent and a second stent of the two or more self-expanding stents and a second radially expanded portion is provided between the first stent and the second stent of the two or more self-expanding stents.

7. The method of claim 1, wherein the stent delivery device further comprises a push sheath and wherein sequentially deploying the two or more self-expanding stents in an overlapping arrangement at the target site in the vessel comprises:
retracting the sheath relative to the elongate member to a position proximal of a first self-expanding stent of the two or more self-expanding stents to deploy the first self-expanding stent;
advancing the sheath relative to the elongate member;
advancing the push sheath relative to the elongate member and sheath to slide a second self-expanding stent of the two or more stents with the first stent along the elongate member; and
retracting the sheath relative to the elongate member to a position proximal of the second self-expanding stent to deploy the second self-expanding stent in an overlapping arrangement with the first self-expanding stent.

8. The method of claim 7, wherein the elongate member includes one or more radially expanded portions disposed between the two or more self-expanding stents, the one or more radially expanded portion being slidable along the elongate member.

9. A stent delivery system, comprising:
a delivery member having a proximal region and a distal region;
a first self-expanding stent disposed around a portion of the distal region of the delivery member in a radially contracted configuration;
a second self-expanding stent disposed around a portion of the distal region of the delivery member in a radially contracted configuration, wherein the first self-expanding stent and the second self-expanding stent are disposed in tandem with the first self-expanding stent distal of the second self-expanding stent; and
a sheath slidably disposed around the delivery member, the first self-expanding stent, and the second self-expanding stent, wherein the sheath is retractable relative to the delivery member to deploy the first self-expanding stent and the second self-expanding stent.

10. The stent delivery system of claim 9, wherein the delivery member includes a first radially expanded portion between the first self-expanding stent and the second self-expanding stent.

11. The stent delivery system of claim 10, wherein the delivery member includes a second radially expanded portion proximal of the second self-expanding stent.

12. The stent delivery system of claim 11, wherein the delivery member includes a third radially expanded portion distal of the first self-expanding stent.

13. The stent delivery system of claim 9, wherein the delivery member includes a distal tip.

14. The stent delivery system of claim 13, wherein the first self-expanding stent is disposed around the distal tip of the delivery member and extends distally thereof.

15. The stent delivery system of claim 9, wherein the delivery member is a guidewire.

16. The stent delivery system of claim 9, wherein the delivery member is a tubular member.

17. The stent delivery system of claim 9, further comprising a second sheath slidably disposed around the delivery member, wherein the second sheath is configured to engage the second self-expanding stent and push the second self-expanding stent along the delivery member.

18. The stent delivery system of claim 10, wherein the first radially expanded portion is slidable relative to the delivery member.

19. A stent delivery system, comprising:
a delivery wire having a proximal region and a distal region, wherein the delivery wire includes a distal tip;
a first self-expanding stent disposed around a portion of the distal region of the delivery wire in a radially contracted configuration, wherein the first self-expanding stent is disposed around the distal tip and extends distally of the distal tip;
a second self-expanding stent disposed around a portion of the distal region of the delivery wire in a radially contracted configuration, wherein the second self-expanding stent is disposed proximal of the first self-expanding stent; and
a sheath slidably disposed around the delivery wire, the first self-expanding stent, and the second self-expanding stent, wherein the sheath is retractable relative to the delivery wire to deploy the first self-expanding stent and the second self-expanding stent.

20. The stent delivery system of claim 19, wherein the delivery wire includes a first radially expanded portion between the first self-expanding stent and the second self-expanding stent.

21. The stent delivery system of claim 20, wherein the delivery wire includes a second radially expanded portion proximal of the second self-expanding stent.

22. The stent delivery system of claim 19, wherein the delivery wire is a guidewire.
